SEQUENCE LISTING (3 sequences)

SEQ ID NO: 1 - protein, 12 aa, Artificial Sequence
  linker sequence
TSGSGSGSGSGT

SEQ ID NO: 2 - DNA, 22 bp, Artificial Sequence
  primer
cacaccagtgtaagggatgttt

SEQ ID NO: 3 - DNA, 22 bp, Artificial Sequence
  primer
cctttagtgatgaagggtaaag

(19) United States
(12) Patent Application Publication
(10) Pub. No.: US 2015/0031583 A1
(43) Pub. Date: Jan. 29, 2015

(54) PHAGE DISPLAY SYSTEM

(71) Applicants: Roderick Slavcev, Waterloo (CA); Jessica Nicastro, Waterloo (CA)

(72) Inventors: Roderick Slavcev, Waterloo (CA); Jessica Nicastro, Waterloo (CA)

(21) Appl. No.: 14/260,732

(22) Filed: Apr. 24, 2014

Related U.S. Application Data
(60) Provisional application No. 61/815,467, filed on Apr. 24, 2013.

(51) Int. Cl. C12N 15/10; C12N 7/00; C12N 15/78; C12P 21/00; C12N 15/70; C12N 15/74

(52) U.S. Cl. CPC C12N 15/1037; C12N 15/70; C12N 15/74; C12N 15/78; C12P 21/00; C12N 15/1093; C12N 7/00; C12N 2795/00041; C12N 2795/00022
USPC 506/14; 435/252.33; 435/252.3; 435/252.34; 435/69.1

(57) ABSTRACT

A phage display system is provided comprising a mutant phage-infected host cell adapted to express a peptide of interest fused to a phage capsid protein, wherein the mutant phage includes a nonsense mutation which prevents expression of the capsid protein as a functional protein, and wherein expression of the peptide of interest is controlled by an inducible repressor and by a suppressor that suppresses the nonsense mutation.

### CROSS-REFERENCE TO RELATED APPLICATION

This application claims priority to U.S. provisional application, 61/815,467, filed Apr. 24, 2013.

### FIELD OF THE INVENTION

The present application relates generally to a phage display system, and more particularly, to a controlled phage display system.

### BACKGROUND OF THE INVENTION

The small-size and the enormous diversity of variants that can be fused to the bacteriophage capsid make bacteriophage ideal candidates for many applications across all industries including targeted therapy and detection in medicine to conjugation with macromolecules, plant science and nanoparticles in materials science. Bacteriophage can also be made in mass quantities very quickly and at a relatively low-cost. The potential for phage as gene delivery vectors is strong since bacteriophage have many of the desirable properties of both the viral and non-viral systems with few of the drawbacks.

Phage display development has undergone considerable growth since EcoRI endonuclease was successfully displayed between the domains of pIII, the filamentous phage minor coat protein. Modern phage display practices with M13 will typically utilize the N-terminals of coat proteins pIII and pVIII, although, proteins have been successfully displayed on all five major capsid proteins. While filamentous phage display systems have contributed greatly to the field, lytic-based phage display systems, notably bacteriophages Lambda (λ), T4, and T7, offer more flexible alternatives that can display both hydrophilic and toxic fusion proteins. Fully formed, bacteriophage lambda has a linear dsDNA genome harboured within an icosahedral capsid comprised of major capsid proteins gpE and gpD that assemble the head in two main steps: prohead assembly followed by DNA packaging. Here, the assembled lambda prohead shell is composed mainly of gpE, in approximately 415 copies, and DNA packaging requires a conformational change to the prohead via the addition gpD to the prohead. The addition of gpD then occurs in 405-420 trimer-clustered molecules that act to increase the head volume and stability and is essential for the packaging of a full-length λ genome. In contrast, gpD deficient viruses can package up to 82% of the wildtype genome; although, they will have to be stabilized by magnesium ions and are extremely sensitive to EDTA.

Since initial λ phage display fusions to the gpV major tail, phage display fusions have since moved onto expression on gpD which confer a high decoration capacity per phage particle (up to 420), and provide a selectable phenotype as a conditionally required packaging protein for full length λ genome. The gpD protein assembles in trimers that are incorporated as prominent protrusions on the surface of the phage capsid making them more accessible for binding to external target molecules. Furthermore, gpD fusions of various sizes have been successfully fused to both the amino and carboxy termini of the protein suggesting that the display of the fusions does not jeopardize the function of the bacteriophage nor prevent fusion proteins from binding the capsid. The C-terminus is generally more tolerant of fusions since the N-terminus is located closer to the 3-fold axis of the gpD trimer and may be involved in gpE interactions. Upon displaying scFV antibodies to both the N and C-termini of gpD, N-terminal fusions were found to impart low recombinant protein loading compared to the C-terminal fusions (50% compared to 88%).

Issues with λ display limit the efficiency and utility of this powerful system. In particular, capsid fusions often interfere with lambda phage morphogenesis, where a positive charge close to the signal sequence cleavage site or a large protein domain may impede capsid assembly. An approach to overcome this limitation is the use of dual expression systems. Originally developed for filamentous phage systems, dual expression systems in λ display first employed amber-suppression mediated control of gpD fusions. The original display system expressed gpD-fusions to N or C-termini in the presence of unfused gpD alleles varied by suppressor tRNA translation of Dam15 in the E. coli host. A plasmid-based dual expression system employing two independently selectable plasmid vectors for wild-type D and the D::fusion, expressed from gpD-λ lysates was later developed by which problematic protein fusions such as that of fibronectin type III could be overcome due to lower levels of interference. They further expanded this approach to incorporate fusions on both the gpD head and gpZ tail proteins, expressing both with high copy number. Another variation includes the use of a plasmid encoding a genomic copy of gpD with an amber mutation at the 5′ end of the gene and another copy of the gene under the control of an inducible promoter, where phage grown on amber suppressors incorporated gpD expressed from the genomic copy and the recombinant gpD from the inducible copy. Most recently, a fusion gene was placed under the control of a temperature sensitive promoter, allowing for repression by growing the bacterial cells at lower temperature, keeping the plasmid encoding the fusion gene repressed until the expression of the plasmid was needed, providing varied fusion decoration levels in resultant phage.

While virtually limitless in application, lytic phage display does not come without limitations, particularly when considering the size and copy number of the displayed peptides as a result of the current display system design. The phage display system practice will need the optimization of fusion coat proteins to wild-type ones since a high ratio of fusion protein may lead to the inefficient assembly of phage particles and depending on the application a low ratio may not elicit the desired results. Accordingly, there is a need for a modified phage display system that overcomes at least one disadvantage of previous such systems.

### SUMMARY OF THE INVENTION

A novel phage display system has now been developed which is adapted to provide controlled peptide delivery, and incorporates mechanisms that provide two-dimensional control over peptide expression and delivery.

Accordingly, in one aspect, a phage display system is provided comprising a mutant phage-infected host cell adapted to express a peptide of interest fused to a phage capsid protein, wherein the phage includes a nonsense mutation which prevents expression of the capsid protein as a functional protein, and wherein expression of the peptide of interest is controlled by an inducible repressor and by a suppressor that suppresses the nonsense mutation.

In another aspect of the invention, method of expressing a peptide of interest in a host cell is provided comprising the steps of: 1) introducing a mutant phage adapted to express the peptide fused to a phage capsid protein to the host cell, wherein the phage includes a nonsense mutation which prevents expression of the capsid protein as a functional protein, and wherein expression of the peptide of interest is controlled by an inducible repressor and by a suppressor that suppresses the nonsense mutation, and 2) culturing the host cell under conditions that permit expression of the peptide of interest.

These and other aspects of the invention are described in the detailed description by reference to the following figures.

### BRIEF DESCRIPTION OF THE FIGURES

FIG. 1. Mean fluorescent event counts for phage preparations on Sup−, SupD, SupE and SupF strains carrying pD::eGFP, cultured at 30, 35, 37, and 39° C. Samples were standardized for titre and diluted 10 fold with PBS to avoid coincidence. Fluorescent events are those that had a fluorescence value greater than 1 (the logarithmic scale equivalent of 0). Error bars represent 95% confidence intervals around the means, calculated from triplicate measurements. In most cases, the error bars are smaller than the size of the corresponding data point.

FIG. 2. 2D density distribution of fluorescence versus side scatter for phage preparations on Sup−, SupD, SupE and SupF strains carrying pD::eGFP, cultured at 30, 35, 37, and 39° C. The density distribution at each temperature is scaled to a constant height. Contour lines represent fractions of maximum density (density quantiles ranging ranging from 0.10 to 0.95 in intervals of 0.05). Events with fluorescence or side scatter values of 1 were excluded. Samples were standardized for titre. SupD and Sup− samples cultured at 37° C. had high concentrations that resulted in a considerable amount of coincidence. The contours corresponding to these samples were calculated from samples diluted 10 fold with PBS; all other contours were calculated from undiluted samples to avoid excessive noise.

FIG. 3. Separation of decorated phage preparations based on fluorescence emitted and size as determined by DLS. Fluorescence and size ratios based on comparison to λF7 grown on W3101 supE [pD::eGFP]. The measured sizes are reported using a % intensity distribution. Each data point was automatically repeated in triplicate, and the average is reported. Sizing results are expressed based on “x” increase compared to wild type, which is λimm21Dam15 (λF7) grown on the BB4 providing gpD_{wt} incorporation into the resultant phage capsid. Measured fluorescence data was analyzed using the SoftMaxPro V5 software based on 6 readings and the samples were run in duplicate with the average being reported. Phage samples were run alongside an eGFP standard to determine the protein concentrations of each sample. Phage fluorescence for each preparation derivative was interpolated from the trend line for known eGFP concentrations.

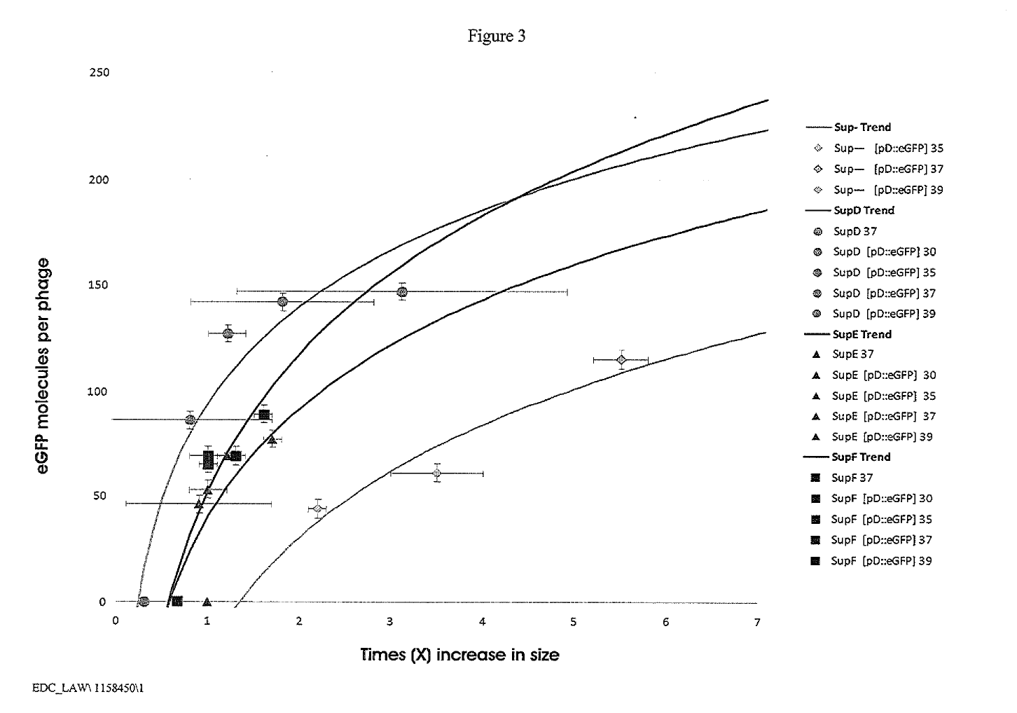

### DETAILED DESCRIPTION OF THE INVENTION

A phage display system is provided comprising a phage-infected host cell adapted to express a peptide of interest fused to a functional phage capsid protein, wherein the phage includes a nonsense mutation which prevents expression of the capsid protein as a functional protein, and wherein expression of the peptide of interest is controlled by an inducible repressor and by a suppressor that suppresses the nonsense mutation.

The present system utilizes host cells that are suitable for phage infection and otherwise suitable for use in recombinant technology. Examples of suitable host cells include bacterial cells, for example, bacterial cells such as E. coli cells, Enterobacter sp., Pseudomonas sp. and Klebsiella sp.

The host cell is infected with a phage adapted to express a peptide of interest fused to a functional phage capsid protein. The phage may be modified to incorporate nucleic acid encoding the peptide-capsid protein fused product within its genome, or may incorporate an expression vector prepared to encode the fused product. Well-established recombinant technology is used to prepare the phage to express the fused product.

The phage is a mutant phage that includes a nonsense mutation which prevents expression of the selected endogenous capsid protein as a functional protein. Expression of functional capsid protein is prevented by inclusion of a nonsense mutation within the capsid-encoding region in the phage genome. Nonsense mutations include, for example, amber mutations, ochre mutations and opal mutations. Such mutant phages can readily be prepared using well-established techniques in the art, such as recombineering, to modify the phage genome to yield a stop codon within the nucleic acid encoding the selected capsid protein. Examples of suitable phage include, for example, filamentous phage such as M13, lytic phages such as bacteriophage lambda, T4, g10 T7 and other lambdoid phages. The selected capsid protein will depend on the phage used, e.g. pIII or pVIII of M13, gpE or gpD of lambda phage, hoc or sac of T4, and g10 of T7 phages. Phage mutants include, for example, amber mutants which possess a premature UAG or TAG codon in the RNA or DNA phage genome, respectively, within the capsid-encoding region; ochre mutants which include a premature UAA or TAA codon in the RNA or DNA phage genome, respectively, within the capsid-encoding region; and opal mutants which include a premature UGA or TGA codon in the RNA or DNA phage genome, respectively, within the capsid-encoding region.

As a first control mechanism for the expression of the peptide of interest, expression of the fused product is under the control of any regulated or inducible promoter, i.e. a promoter which is activated under a particular physical or chemical condition or stimulus. Examples of suitable promoters include thermally-regulated promoters such as the λ pL promoter, IPTG regulated lac promoter, the glucose regulated ara promoter, the T7 polymerase regulated promoter, cold-shock inducible cspA promoter, a heat shock inducible hsp promoter, pH inducible promoters, or combinations thereof, such as tac (T7 and lac) dual regulated promoter. Thus, expression of the peptide of interest is controlled by altering the physical or chemical stimulus to achieve the desired degree of peptide expression. For example, for thermally-regulated promoters, optimal expression may be achieved at 37° C., while reduced expression is achieved at lower temperatures. Similarly, optimal expression using a pH inducible promoter may be achieved at an acidic pH, while reduced expression is achieved as the pH is increased.

As a second control mechanism for the expression of the peptide of interest, the host cell encodes a suppressor (e.g. a sup+ strain) that suppresses the nonsense mutation within the mutant phage and permits the phage to “read through” the nonsense stop codon to produce a functional capsid protein. The suppressor strain is selected to correspond with the nonsense mutation, as well as being selected based on the degree of suppression of the nonsense mutation that it provides. For example, in E. coli, suppressor strains, supE, supE, supD, supU, supF and supZ suppress amber mutations, while suppressor strains, supB, supL, supN, supC and supM suppress ochre mutations, and glyT and trpT strains suppress opal mutations. Temperature-sensitive alleles of these strains may also be used. Varying degrees of suppression are provided by each suppressor to yield varying degrees of decoration, and thus expression, of the peptide of interest. For example, in nucleic acid encoding the glycoprotein D capsid protein in lambda phage, suppressor E fully suppresses an amber mutation restoring the native sequence of glycoprotein D, while suppressor F partially suppresses the amber mutation and suppressor D provides little or no suppression of an amber mutation.

As one of skill in the art will appreciate, the peptide of interest to be expressed is not limited to any particular peptide or protein. The term “peptide” is used herein to encompass both partial and full protein sequences. In one embodiment, the peptide may, for example, be selected for therapeutic utility. For example, the peptide may be ornithine transcarbamylase for use in the treatment of a disease condition such as ornithine transcarbamylase deficiency (OTCD). The vector may also be used to deliver a peptide vaccine such as an HIV Env-Gag VLP; or a molecular adjuvant such as I1-12; or a prodrug activating enzyme such as thymidine kinase in the treatment of various cancers. In other embodiments, the peptide may be selected for use in material sciences, for example in biosurfactant design, for use industrially or agriculturally.

The present phage display system may further be modified to express additional peptides of interest, i.e. a multi-valent system, the expression of which may also be under the control mechanisms of the system as set out above.

Embodiments of the invention are described by reference to the following specific examples which are not to be construed as limiting.

### Example 1

The following is a description of the methods and materials employed.

Lambda phages, E. coli K-12 strains and plasmids used in this work are shown in Table 1 below.

TABLE 1
Bacteria, phage and plasmids

| Cell/Phage/ | | |
|---|---|---|
| Plasmid | | Source/ |
| Designation | Genotype | Reference |
| Bacterial | | |
| Strains | | |
| BB4 | supF58 supE44 hsdRS14 galK2 | Agilent |
| | galT22 trpR55 metB1 tonA | Technologies, |
| | DE(lac) U169 | Inc. |
| DS-3 | F-, phoA4(Am), serU132(AS), | CGSC# 4604 |
| | his-45, rpsL114(Str^{R}), | Hoffman & |
| | valR116 | Wilhelm (1970) |
| W3899 | F-, glnV44(AS), nadB7 | CGSC# 5177 |
| | | Edlin & Sundaram |
| | | (1989) |
| K1227 | F-, ompF627(T2R), | CGSC# 7058 |
| | tyrT5888(AS), | |
| | oppC506::Tn10, | |
| | fadL701(T2R), relA1, | |
| | pitA10, spoT1, rrnB-2, | |
| | mcrB1, creCS10 | |
| CAG12077 | F-, crcA280::Tn10, λ-, | CGSC# 7347 |
| | rph-1 | Grossman et al. |
| | | (1989) |
| | | Nichols et al. |
| | | (1998) |
| CAG12156 | F-, λ-, uvrC279::Tn10, | CGSC# 7394 |
| | rph-1 | Grossman et al. |
| | | (1989) |
| W3101 | F-, galT22, λ-, | CGSC #4467 |
| | IN(rrnD-rrnE)1, rph-1 | Bachmann (1972) |
| W3101 SupD | F-, galT22, λ-, | This study |
| | IN(rrnD-rrnE)1, rph-1, | |
| | uvrC279::Tn10, serU132(AS), | |
| W3101 SupE | F-, galT22, λ-, | This study |
| | IN(rrnD-rrnE)1, rph-1, | |
| | crcA280::Tn10, glnV44(AS), | |
| W3101 SupF | F-, galT22, λ-, | This study |
| | IN(rrnD-rrnE)1, rph-1, | |
| | oppC506::Tn10, tyrT5888(AS) | |
| Phage | | |
| Strains | | |
| λimm21 | λimm21clts | Windass & |
| | | Brammar (1979) |
| λF7 | λDam15imm21clts | Mikawa et al. |
| | | (1996) Maruyama |
| | | et al. (1994) |
| Plasmids | | |
| pPL451 | pM-cI857-pL-cI857-pL-MCS-tL | National |
| | | BioResource |
| | | Project (NBRP); |
| pPL451 gpD | pM-cI857-pL-cI857-pL-D-tL | Stokolenko et |
| | | al. (2012) |
| pPL451 | pM-cI857-pL-cI857-pL-D::gfp-tL | Stokolenko et |
| gpD::eGFP | | al. (2012) |

Amber suppressor strains (SupD, E, F) of W3101 were constructed in 2 steps. First, P1 rev6-mediated transduction of a tetracycline resistance (TcR) marker from CAG12077 to recipient amber suppressor (AS) strains DS-3, and W3899 was performed to link the AS marker to the TcR marker. K1227 (SupF) already possessed a linked TcR marker (see Table 1). P1 rev6 was used to cotransduce the AS and TcR marker (<2 min distance) into recipient W3101 cells, which were screened with λimm21Dam15 (λF7) and λSam7 phage to ensure transfer of the AS alleles, respectively linked to the TcR marker, derived from donor strains, to the W3101 non-suppressor (Sup−) recipient strain. The construction of plasmid pPL451-gpD::eGFP (herein referred to as pD::eGFP) was previously described (Sokolenko et al. 2012. Cytometry. Part A: The Journal of the International Society for Analytical Cytology, pp. 1-9), where gpD::eGFP expression from this multicopy plasmid (pPL451) is governed by a temperature-sensitive allele of the λ C1857 repressor. The D::eGFP sequence designed creates a C-terminal eGFP translational fusion with λ D capsid gene, separated by an in-frame short linker encoding amino acids (TSGSGSGSGSGT) (SEQ ID NO:1) followed by a KpnI cut site to allow for removal and exchange of eGFP.

Plasmid pPL451-gpD (herein referred to as pD) was constructed by digesting pPL451-gpD::eGFP by KpnI removing all but the last 30 C-terminal by of eGFP. Plasmid pPL451 was digested with HpaI and pPL451 gpD::eGFP and pPL451 gpD were double digested with HpaI and NcoI and the digestion pattern analyzed to ensure cloning accuracy.

### Phage Amplification and Purification

Cultures of transformed W3101 Sup+/Sup− [pPL451gpD:eGFP] (pD::eGFP) Escherichia coli cells were grown on plates at 30, 35, 37 or 39° C. overnight, while cultures of W3101 [pPL451gpD] (pD) were grown up at 37° C. only, prior to the addition of primary lysate dilutions. 1:10 dilutions of primary lysates were prepared in 1 mL of TN buffer (0.01 M Tris-HCl and 0.1 M NaCl, pH 7.8, (Fisher Scientific, USA). Lysate dilutions were added to 0.3 ml of cells, incubated for 2 hr at experimental temperature prior to adding 3 ml of top agar (Bacto Tryptone and Bacto Agar from Difco Laboratories, Sparks, Md.) and plates were incubated overnight at the temperature that the experimental temperature. Plate lysates were then prepared by adding 10 mL of TN buffer to the surface of the plate, incubating for 8 hr at 4° C., then transferring solution and top agar to a conical tube, mixing and centrifuging at 12K RPM (Avanti J-E Centrifuge, Beckman Coulter, Mississauga Canada) at 4° C. for 20 min. The supernatant was poured into a fresh ice-cold (0° C.) conical tube and lysates were then precipitated for purification and concentration purposes with 20% polyethylene glycol (PEG)-8000 (Fisher Scientific, USA), 2.5 M NaCl using a standard protocol and was resuspended in fresh TN buffer. To remove cellular debris, lysates were then filtered through a sterile 0.45 μM syringe filter (BD Discardit, India). To purify lysates from unincorporated fusion and other cellular proteins, particularly unincorporated gpD::eGFP, lysates were purified as previously described (Sain and Erdei. Anal Biochem. 1981 Jan. 1; 110(1):128-30) by gel chromatography, offering lysate purity comparable to that of CsCl centrifugation and amenable to lysate smaller volumes. Briefly, lysates were passed through a 50-150 μL agarose size exclusion column (4% beads, ABT, Spain) in buffer containing 10 mM Tris-HCl (pH 7.5) and 1 mM MgCl2. Phage were titered at each step of purification by standard viability assays on fresh Sup+ BB4 (supE, supF) E. coli cells, with final phage titers ranging from 10¹⁰ to mid 10¹¹ phage/ml. Samples were stored at 4° C.

### Sequencing of the λ Dam15 Mutation

The λ Dam15 mutation was amplified from λ F7 (λ imm21Dam15) using the primers (F) 5′CACACCAGTGTAAGGGATGTTT-3′ (SEQ ID NO:2); and (R) 5′ CCTTTAGTGATGAAGGGTAAAG-3′ (SEQ ID NO:3) (Sigma-Aldrich, Canada). The 330 bp amplified allele was purified and sequenced at York University, North York on an Automated DNA Sequencing Facility on an Applied Biosystems 3130xL DNA Sequencer.

### Phage Titration and Efficiency of Plating Assays

Viable counts of phage were quantified by standard plaque forming unit assay using BB4 cells (SupE, SupF double suppressor) as the 100% control as this strain repeatedly generates highest titers of λF7. Plates were incubated overnight at experimental temperature when necessary, otherwise, at 37° C. Relative plating efficiency at all test temperatures was determined by measuring phage titer on experimental strain divided by that scored on BB4.

### Immunoblotting

Immunoblot experiments were conducted using rabbit anti-gfp polyclonal antibody (gift from Dr. B. Moffatt, Waterloo). Samples were denatured by boiling for 10 min, then placed on ice and Centrifuged at 12K RPM for 1 min. A total volume of 20 μl was run with a range of 100-200 ng of total protein being run. Samples were run alongside a GFP standard ranging from 10 pg/μl to 100 ng/μl as positive standard, then separated by 15% SDS-PAGE. After electrophoresis the gel was placed in transfer buffer (48 mM Tris, 39 mM glycine, 20% (v/v) methanol, 0.04% (w/v) SDS, (pH 9 to 9.4) for 10 min. The protein was transferred to a nitrocellulose membrane at 20 V for 45 min. The membrane was then stained with Ponceau S stain (0.2% w/v) by shaking for 10 min and de-staining in dH2O until bands were visible. The stain was then removed by shaking in 1× PBS buffer until the stain (and bands were no longer visible). The membrane was then placed in PVA for 30 seconds to block, then washed in PBS/milk/Tween for 10 min. The membrane was then incubated at 4° C. overnight in PBS/milk/Tween plus 1/30th dilution of primary rabbit anti-GFP antibody. After incubation the membrane was washed three times in PBS/milk/Tween before adding the secondary AP conjugate antibody (anti-rabbit) at 1:2000 and incubating for two hrs. The membrane was then rinsed three more times with PBS/milk/Tween for ten min before a final rinse in PBS for five min. The membrane was visualized by a Typhoon imaging system. A second membrane was blocked with TBST for one hour at room temperature and incubated with primary antibody (1:30) for one hour at room temperature and washed three times for 10 min with TBST and then incubated with a different secondary antibody in TBST for one hour at room temperature and then washed again three times and the bands were detected with SuperSignal West Pico Chemiluminescent substrate and visualized on a Kodak imaging system.

### Dynamic Light Scattering of Phage

Phage particle size was measured at 25° C. using a Malvern Zetasizer Nano ZS instrument (Malvern instruments, UK). Samples were prepared in Milli-Q water and filtered using a 100 nm filter prior to measurement. The measured sizes are reported using a % intensity distribution. Each data point was automatically repeated in triplicate, and the average was reported. Sizing results are expressed based on “x” increase compared to wild type, which is λimm21Dam15 (λF7) grown on the BB4 providing gpDwt incorporation into the resultant phage capsid.

### Whole Phage Fluorimetric Analysis

Phage samples were prepared in TN buffer by diluting each sample to a uniform concentration of 2.0×109 PFU/mL. 150 μL of each prepared sample was then added to a sterile 96 well plate (Starstedt) and were analysed using a SpectraMax M5 spectrophotometer at an excitation of 485 nm and an emission of 555 nm. The data was analyzed using the SoftMaxPro V5 software where each well was set to be automatically read 6 times and the samples were run in duplicate with the average being reported. Phage samples were run alongside an eGFP standard (Cell Biolabs Inc. #212103) to determine the protein concentrations of each sample. Phage fluorescence for each preparation derivative was interpolated from the trend-line for known eGFP concentrations. The standard error of each of the sample fluorescence values was done in a weighted analysis against the determination of the sum of the squares of the fluorescence based deviations from the trendline curve of the fluorescent standard. The standard deviation of each sample was not taken into consideration in the calculations as these deviations were determined to be statistically less significant than the determination from the interpolation itself.

### Flow Cytometry Analysis

Fluorescence and side scatter of phage samples were measured on a FACSCalibur flow cytometer (BD Biosciences, San Jose, Calif.). Briefly, the flow cytometer was equipped with a 15 mW air-cooled argon-ion laser, with an excitation frequency of 488 nm. Side scatter (SSC) and Fluorescence (FL) photomultiplier tube (PMT) voltages were set to 500 V and 525 V, respectively, with logarithmic amplification. A 530/30 nm bandpass filter was used for the observation of D::eGFP fluorescence. All samples were serially diluted 1×, 10×, and 100× using phosphate buffer saline (PBS) and run for 30 seconds at the low flow setting (20 μL/min). The dilutions were used to assess instances of “coincidence”, where a high sample concentration results in multiple particles being observed as a single event. More information on how the instrument settings were chosen and the results of negative controls are described by Sokolenko et al. (2012). All data analysis was performed by in-house scripts written in the R programming language. Samples were compared based on the number of observed fluorescent events (those with FL values greater than 1—the logarithmic scale equivalent of 0) and the distribution of their SSC and FL values. Contour plots of SSC and FL values were generated using kernel density estimation. All plots were created using the ggplot2 package.

### EDTA Sensitivity

Phage were diluted in TN buffer (0.01 M Tris-HCl and 0.1 M NaCl, pH 7.8) (Fisher Scientific, USA) to a universal concentration of 4.0×108 PFU/mL then were diluted 100-fold into TN/EDTA (0.01 M Tris-HCl (pH7.8), 0.1 M NaCl, and 0.01M EDTA) and incubated for 25 minutes at room temperature. EDTA inactivation was stopped by diluting the samples 100-fold into TN buffer and immediately plating the mixture on BB4 cells (SupE, SupF double suppressor). Plates were incubated overnight at 37° C. Relative plating efficiency of each sample was determined by measuring phage titer divided by the original phage titers as the 100% control.

### Results

### Sequencing the Dam15 Mutation and Suppressor Conferred Alleles

Despite the common use of the λF7 (λimm21Dam15) derivatives in phage display, the exact positioning of this mutation has to date never been elucidated. The 333 by Dam15 allele was first sequenced to determine that the amber mutation was localized to the 204th by of D, converting the 68th CAG codon (glutamine), to an amber translational stop signal (TAG) codon. In a Sup− cell the amber mutation imparts a premature translational stop resulting in a truncated 68 a.a. non-functional gpD fragment that is incapable of stabilizing and packaging full length λ DNA. Growing λ Dam15 phage on an amber suppressor host may result in a phenotypic amino acid substitution of glutamine residue at position 68. Only SupE strains will code glutamine at the 68th codon restoring the pristine sequence of gpD, while SupD will confer a serine substitution, yielding gpDQ68S and SupF will confer a tyrosine substitution, yielding gpDQ68Y. To identify the ability of different amber suppressors to reverse the lethal Dam15 mutation, an isogenic set of amber suppressor derivatives of W3101 (Sup−) were generated and the plating efficiency of λimm21Dam15 was determined (Table 2),

TABLE 2
Variable amber suppression and complementation of the Dam15 mutation

| Strain ¹/ | Relative efficiency of plating (eop) ³ | | | | |
|---|---|---|---|---|---|
| Plasmid ² | 30° C. | 32-33° C. | 35° C. | 37° C. | 39-40° C. |
| Sup− | Nd | Nd | nd | 1.92 × 10⁻⁶ | nd |
| pD⁻ | 4.06 × 10⁻⁶ | 5.0 × 10⁻⁶ | 3.1 × 10⁻⁵ | 1.7 × 10⁻⁶ | 2.0 × 10⁻⁶ |
| pD⁺ | 7.5 × 10⁻⁶ | 3.2 × 10⁻⁵ | 2.1 × 10⁻⁴ | 0.15 | 0.93 |
| pD::eGFP | 7.5 × 10⁻⁶ | 2.0 × 10⁻⁵ | 2.0 × 10⁻⁵ | 0.02 | 0.01 |
| SupD | Nd | Nd | nd | 1.13 × 10⁻⁵ | nd |
| PD⁻ | 8.8 × 10⁻⁵ | 6.5 × 10⁻⁵ | 1.5 × 10⁻⁵ | 9.5 × 10⁻⁶ | 2.3 × 10⁻⁵ |
| pD::eGFP | 4.9 × 10⁻⁵ | 1.8 × 10⁻⁵ | 8.0 × 10⁻⁵ | 0.09 | 0.05 |
| SupE | Nd | Nd | nd | 0.09 | nd |
| pD⁻ | 0.04 | 0.11 | 0.07 | 0.03 | 0.08 |
| pD::eGFP | 0.1 | 0.04 | 0.02 | 0.07 | 0.03 |
| SupF | Nd | Nd | nd | 0.19 | nd |
| pD⁻ | 0.01 | 0.01 | 0.04 | 0.04 | 0.03 |
| pD::eGFP | 0.1 | 0.13 | 0.06 | 0.07 | 0.05 |

¹ Derivatives are W3101 background.
² Derivatives of CI857 temperature regulated expression plasmid pPL451.
³ All eop determinations from a minimum of three assays and determined using BB4 (SupE, SupF) as the 100% control.

Of the three suppressors, the SupD host, yielding gpDQ68S, was least effective at reversing the Dam15 mutation, improving viability by only 10 fold compared to that of the Sup− control at 37° C., and generated pinpoint plaques, indicative of a very low burst size of viable progeny. In contrast, the SupE host that restores the pristine sequence of gpD upon mistranslation of Dam15, restored viability to about 10% that of the double suppressor (SupE, SupF) positive control. The SupF host, conferring the gpDQ68Y allele performed as well as (if not marginally better than) SupE in an otherwise isogenic host background. SupF restored viability to about 20% that of the positive control, despite the size and polarity difference between glutamine (SupE) and tyrosine (SupF). These results were corroborated by the efficiency of plating (eop) of the strains carrying the parent (backbone) plasmid, pPL451, showing that suppressor capability was not dramatically impacted by differences in temperature, not by the presence of the temperature-regulated expression parent plasmid (Table 2).

The capacity for the pD and pD::eGFP plasmids, in which the expression of the D allele is governed by the temperature sensitive λ CI857 repressor, was determined. The nonsuppressor strain W3101 was transformed by pD and pD::eGFP given that the ability to properly package and produce viable λDam15 particles would rely solely upon in trans complementation for the Dam15 mutation from the plasmid. As expected, at increasing temperatures, complementation for Dam15 by plasmid-borne D increased (as repressor activity decreased), with optimal results seen at 39° C. (Table 2) where complementation restored near full viability of λF7. The experimental plasmid expressing D::eGFP also showed a temperature-governed complementation profile that paralleled that of the D plasmid, despite that viability was about 10 fold lower at all assayed temperatures, with best complementation achieved again at 39-40° C. In contrast, the D− parent plasmid was unable to complement for the mutation at any tested temperature. Complementation for the Dam15 mutation by the gpD and gpD::eGFP plasmids could not be differentiated from suppression of the mutation on SupE and SupF hosts carrying the plasmid due to strong suppressor activity by these strains at all temperatures. In contrast, due to the low viability of λDam15 on the SupD derivative, complementation by gpD::eGFP was observable as expression of the fusion increased with rising temperature and like the Sup− [pD::eGFP] strain, SupD[pD::eGFP] provided the greatest and a similar level of complementation for the Dam15 mutation at 39-40° C.

Fluorimetric Analysis of gpD::eGFP Decorated Phage

λDam15 phage, variably decorated by gpD::eGFP by passaging through the Sup− and Sup+ strains carrying the pD::eGFP plasmid at various temperatures were standardized for titer and assayed for functional fluorescence by fluorimetry. Fluorescence was interpolated against an eGFP standard of known concentration and the average number of eGFP fusions per phage was determined (Table 3).

TABLE 3
Estimated average eGFP molecules per phage.

| Strain | Estimated eGFP/phage ² | | | |
|---|---|---|---|---|
| (+/−Plasmid) ¹ | 30° C. | 35° C. | 37° C. | 39° C. |
| Sup⁻ | n/a | 44 ± 4.22 | 115 ± 4.39 | 61 ± 4.16 |
| [pD::eGFP] | | | | |
| SupD | 86 ± 4.20 | 127 ± 4.16 | 147 ± 4.18 | 142 ± 4.17 |
| [pD::eGFP] | | | | |
| SupE | 46 ± 4.26 | 53 ± 4.34 | 77 ± 4.22 | 69 ± 4.25 |
| [pD::eGFP] | | | | |
| SupF | 65 ± 4.27 | 69 ± 4.25 | 89 ± 4.19 | 69 ± 4.44 |
| [pD::eGFP] | | | | |

¹ all strains are derivatives of W3101.
² fluorescence measurements were divided by those derived from λF7 grown on that strain. Values in parentheses denote calculated number of functional eGP fusions per phage interpolated from eGFP purified protein fluorescence calibration curve.
³ Phage preparation on W3101[pD] at 37° C. expressing gpD_{wt} in trans

Due to the lack of expression of a functional gpD allele from either the phage or the plasmid at 30° C. in Sup− [pD::eGFP], a lysate could not be generated under this condition. However, upon raising the temperature to >35° C., phage viability improved and eGFP decoration was increasingly evident with rising temperature (derepressing expression of D::eGFP) beyond this point, with highest fluorescence observed at 37° C. All lysate preparations similarly showed increases in fluorescence to 37° C., albeit considerably lower for phage prepared on the strong suppressor strains, SupE and SupF, which might be attributed to preferential packaging of gpDwt and gpDQ68Y over gpD::eGFP proteins during phage capsid assembly. In contrast, lysates prepared on SupD, the weakest suppressor, demonstrated the strongest fluorescence from all preparations, with a notable signal present even at 30° C. The noted signal at this temperature was likely due to leaky expression of D::eGFP from the plasmid, but more importantly demonstrates the strong ability of gpD::eGFP to complement for the poorly functional gpDQ68Y protein. It was also noted that lysates prepared at 39-40° C. indicated decreased fluorescence compared to those prepared at 37° C. on all Sup+ and Sup− strains, despite that derepression of D::eGFP at this temperature is complete and offered highest complementation efficiency for the Dam15 mutation.

Size Comparisons of gpD::eGFP Decorated Phage

Lysates prepared on the Sup series at various temperatures were standardized for titre and sized by dynamic light scattering to approximate relative size differences between undecorated and decorated phage derivatives (Table 4).

TABLE 4
Sizing of λDam15 phage variably decorated by gpD::eGFP

| | | Times (X) increase in phage diameter² | | | |
|---|---|---|---|---|---|
| Strain [+Plasmid] ¹ | gpD Allele | 30° C. | 35° C. | 37° C. | 39° C. |
| Sup⁻ | gpD_{wt} | nd | nd | 1.1 ± 0.2 | nd |
| Sup⁻ [pD::eGFP] | | n/a | 2.2 ± 0.5 | 5.5 ± 0.8 | 3.5 ± 0.9 |
| SupD | gpD_{wt} | nd | nd | 0.3 ± 0.03 | nd |
| SupD [pD::eGFP] | | 0.8 ± 0.1 | 1.2 ± 0.7 | 3.1 ± 1.5 | 1.8 ± 0.2 |
| SupE | gpDQ68S | nd | nd | 1.0 ± 0.1 | nd |
| SupE [pD::eGFP] | | 0.9 ± 0.2 | 1.0 ± 0.1 | 1.7 ± 0.2 | 1.2 ± 0.2 |
| SupF | gpDQ68Y | nd | nd | 0.67 ± 0.3 | nd |
| SupF [pD::eGFP] | | 1.0 ± 0.3 | 1.3 ± 0.2 | 1.6 ± 0.6 | 1.0 ± 0.3 |

¹ Lysate produced on strain at respective temp. All strains are derivatives of W3101.
² A minimum of three runs of triplicate determinations of size determination from DLS were divided by size determinations for naked phage grown on each of the Sup strains. Comparisons are for phage grown on SupE in absence of complementation.
³ Phage preparation on W3101[pD] at 37° C. expressing gpD_{wt} in trans

Sizing of λimm21Dam15 phage grown on SupE, yielding the gpDwt allele, compared well to λimm21 and was used as the wild type phage size control, generating phage with an average diameter of 62 nm. Relative size differences between λDam15 phage grown on the suppressor host series at 37° C. in the absence of gpD::eGFP, was first determined. As expected, phage grown on Sup− [pD] cells at this inducing temperature were similar in size to the control, while SupD preparations were surprisingly only about a third of the size. This finding may suggest that the gpDQ68S protein incorporates very poorly into the capsid, resulting in the smaller capsid size. Next, λDam15 phage passaged through the suppressors carrying the gpD::eGFP plasmid at various temperatures were sized by DLS in triplicate and compared to the size of the undecorated control phage grown on SupE in the absence of pD::eGFP (Table 4). In all cases, phage size increased as temperature increased from 30 through 37° C., where expression of D::eGFP was increasingly derepressed. Interestingly, phage samples prepared at 39-40° C., where D: eGFP expression should be maximal, indicated a 30 to 40% decrease in phage diameter compared to that at 37° C., suggesting that either fewer gpD::eGFP were incorporating into the capsid, despite complete derepression of D:: eGFP, or eGFP were being shed from the decorated phage. Phage prepared on SupD [pD::eGFP] at 37 and 39° C. were particularly interesting, however, as phage samples indicated two notable size peaks, denoting vastly different diameters at substantial occurrence; a first peak at ˜30 nm and the second at ˜280 nm. The former size is similar to that seen for λF7 passaged through the SupD strain in absence of plasmid. The very large error in average size determination for SupD phage preps at this temperature (Table 4) can be attributed to this bimodal size distribution.

Flow Cytometry of gpD::eGFP Decorated Phage

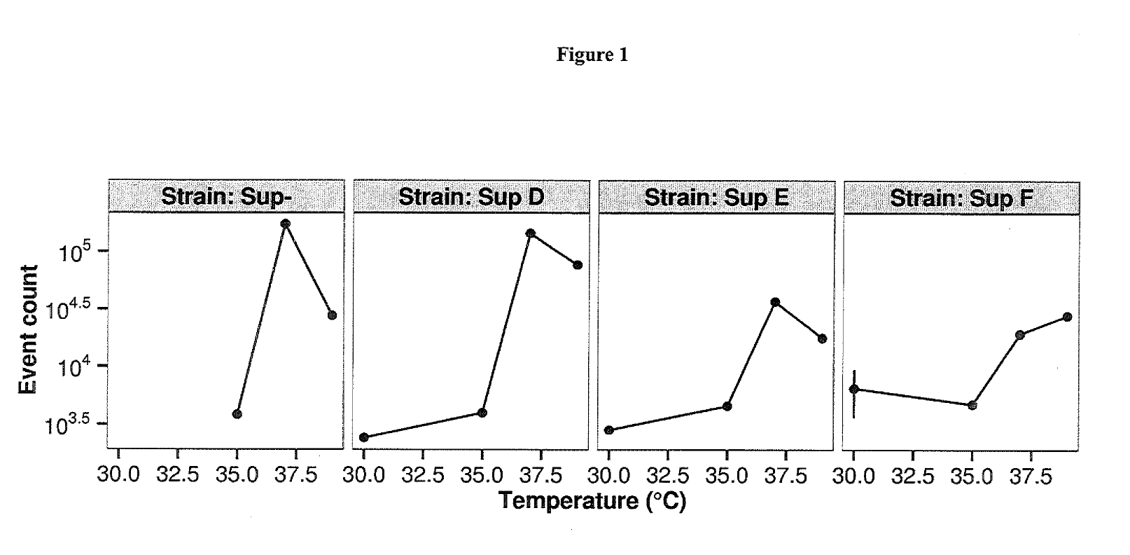

Lysates prepared on Sup strains at various temperatures were standardized for litre and analyzed with a flow cytometer. A sample's side scatter and fluorescence profile was found to vary between phage preparations and to be influenced by the degree of eGFP capsid decoration. This group previously optimized the use of flow cytometry for this application using phage passaged through SupE [pD::eGFP] (Sokolenko et al. 2012). A simple count comparison of fluorescent events was the most basic analysis performed on the data obtained (FIG. 1). An overall increase in the count of fluorescent events was observed as a function of temperature for all strains. The temperature dependence was generally more pronounced between 35 and 37° C. than between 30 and 35° C., especially for SupD and Sup− preparations. For all lysate preparations, the number of fluorescent events evident was an order of magnitude higher between 35° C. and 37° C., in agreement with the CI[Ts]857 temperature-lability profile and subsequent expression of gpD::eGFP. Higher culturing temperatures corresponded well with expected higher fluorescence up to 37° C. due to increased gpD::eGFP expression and decoration at this temperature.

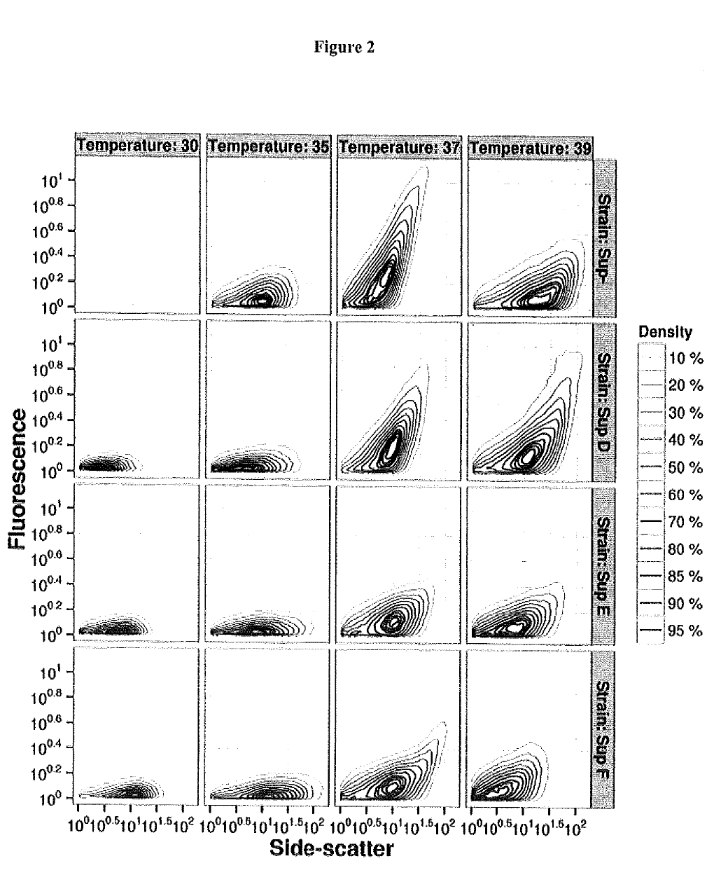

It was previously demonstrated that 2D fluorescence/side-scatter density is a much better visualization tool for discriminating different types of fluorescent events between preparations. Here, this approach was expanded to compare phage preparations on all Sup strains at all experimental temperatures (FIG. 2). A change in temperature from 30° C. to 35° C. corresponded to an increase in only side-scatter. An increase to 37° C., on the other hand, had a greater impact on event fluorescence, as can be seen in the prominent movement of the main cluster away from the x-axis. The low temperature changes in side scatter can be interpreted as the result of initial capsid modification, with the increased expression of D::eGFP at 37° C. being required to generate a significant amount of fluorescence. Consistent with the event count analysis, strong increases in Sup− and SupD fluorescence were noted at 37° C., as compared to the comparatively mild increase in fluorescence observed for lysates prepared on the strong suppressors, SupE and SupF, carrying the gpD::eGFP plasmid. While increasing the culturing temperature to 39° C. did not significantly alter the total number of events detected, it did alter the fluorescence distribution across all preparations, most evident in the decrease of fluorescence for SupD and Sup− strains (FIG. 2). The impact of increasing the expression level of gpD::eGFP by increasing the temperature past 37° C. on the generation of eGFP-tagged phage appears to be limited. Similar to the results obtained for fluorimetry assays, 37° C. again was the upper limit for fluorescence, with evident reductions in fluorescence at 39-40° C. compared to that at 37° C.

Protein Shedding from Phage Preparations

The reduction in phage size and fluorescence noted in phages prepared on the Sup series at 40° C. versus their 37° C. counterparts was further investigated. It was first examined whether or not eGFP were being shed from decorated phage by performing electrophoretic separation, followed by fluorescence analysis on the two species that notes the greatest degree of error for both fluorescence and size determination. SupD [pD::eGFP] preps demonstrated a high degree of eGFP shedding from the decorated capsid as noted by the strong presence of a ˜33 kDa fluorescent protein compared to the ˜43 kDa fluorescent protein visualized for gpD::eGFP. This effect was also seen for the Sup− [pD::eGFP] prep. Interestingly, the band runs slightly higher than that seen for eGFP (˜29 kDa), which may be due to the incorporation of the linker into the cleaved product. As expected, the SupD [pD] prep phage control, showed no fluorescence as D is not fused to eGFP in this plasmid.

### EDTA Resistance of Decorated and Non-Decorated Phage

To assess the functionality of the surface proteins of λDam15 phage, variably decorated by different gpD::eGFP and/or different isotypes of gpD, each of the phage samples grown at 37° C. was tested for resistance to EDTA (Table 5). A remarkable variation in post-treatment survival rates was noted. Lysates prepared on SupE and on Sup− [pD], both conferring the gpDwt allele, had the greatest EDTA resistance with approximately 91% and 93% resistant progeny, respectively. Phage grown on SupF, generating the gpDQ68Y allele, demonstrated a 44% survival rate compared to the wild type, while those grown on SupD, generating the gpDQ68S allele, showed the poorest survival rate of only 35%. The noted difference in survival rate is believed to be attributed to the relatively stable packaging of gpDwt over either of the gpDQ68Y and gpDQ68S alleles, although gpDQ68Y conferred excellent viability to passaged phage.

Phages variably decorated by passaging through Sup+ [pD::eGFP] strains showed relatively consistent survival rates among the samples ranging from 54-59%, albeit a drop in resistance from that seen with the packaging of gpDwt with SupE and an increase in resistance from those packaging the gpDQ68Y allele from SupF and gpDQ68S alleles from SupD. Lysates developed on Sup− [pD::eGFP] had a relatively low survival rate of 36% suggesting that decoration is not complete. The variable display of both the fusion protein with pD::eGFP and the gpD alleles conferred were found to be more stable than either of the gpDQ68Y allele, the gpDQ68S allele or the pD::eGFP fusion alone, while those exhibiting wild-type gpD were found to be the most resistant to EDTA, as expected.

### Discussion

This strategy is based on the combination of two competing genetic principles: 1) Various alleles of gpD based on isogenic suppression of the λDam15 mutation; and 2) Plasmid-borne D::X fusion expression regulated by temperature to complement the Dam15 mutation. It is shown here that through the combination of these two dimensions dramatic variations in phage decoration through gpD::X fusions can be achieved.

1. A phage display system comprising mutant phage-infected host cells adapted to express a peptide of interest fused to a phage capsid protein, wherein the mutant phage includes a nonsense mutation which prevents expression of the capsid protein as a functional protein, and wherein expression of the peptide of interest fused to the capsid protein is controlled by an inducible repressor and by a suppressor that suppresses the nonsense mutation.

2. The phage display system as defined in claim 1, wherein the host cell is a bacterial cell.

3. The phage display system as defined in claim 2, wherein the bacterial cell is selected from the group consisting of E. coli, Enterobacter sp., Pseudomonas sp. and Klebsiella sp.

4. The phage display system as defined in claim 1, wherein the mutant phage is an amber mutant, an ochre mutant or an opal mutant.

5. The phage display system as defined in claim 4, wherein the mutant phage is selected from filamentous phages and lytic phages.

6. The phage display system as defined in claim 1, wherein the capsid protein is selected from the group consisting of pIII, pVIII, gpD, hoc, soc and g10.

7. The phage display system as defined in claim 1, wherein the inducible repressor is a thermally-regulated promoter, an IPTG regulated be promoter, a glucose regulated ara promoter, a T7 polymerase regulated promoter, a cold-shock inducible cspA promoter, a heat shock inducible hsp promoter, a pH inducible promoter or a combination thereof.

8. The phage display system as defined in claim 1, wherein the host cell is an E. coli suppressor strain.

9. The phage display system as defined in claim 8, wherein the suppressor strain is selected from the group consisting of supE, supP, supD, supU, supF, supZ, supB, supL, supN, supC, supM, glyT, trpT and temperature-sensitive alleles thereof.

10. The phage display system as defined in claim 1, wherein the peptide of interest has therapeutic utility.

11. The phage display system as defined in claim 1, modified to express multiple peptides of interest, wherein each peptide is controlled by an inducible repressor and a suppressor.

12. A method of expressing a peptide of interest in a host cell comprising the steps of: 1) introducing a mutant phage adapted to express the peptide fused to a phage capsid protein to the host cell, wherein the phage includes a nonsense mutation which prevents expression of the capsid protein as a functional protein, and wherein expression of the peptide of interest is controlled by an inducible repressor and by a suppressor that suppresses the nonsense mutation, and 2) culturing the host cell under conditions that permit expression of the peptide of interest.

13. The method as defined in claim 12, wherein the host cell is a bacterial cell.

14. The method as defined in claim 12, wherein the mutant phage is an amber mutant, an ochre mutant or an opal mutant.

15. The method as defined in claim 14, wherein the mutant phage is selected from filamentous phages and lytic phages.

16. The method as defined in claim 12, wherein the capsid protein is selected from the group consisting of pIII, pVIII, gpD, hoc, soc and g10.

17. The method as defined in claim 12, wherein the inducible repressor is a thermally-regulated promoter, an IPTG regulated lac promoter, a glucose regulated ara promoter, a T7 polymerase regulated promoter, a cold-shock inducible cspA promoter, a heat shock inducible hsp promoter, a pH inducible promoter or a combination thereof.

18. The method as defined in claim 12, wherein the host cell is an E. coli suppressor strain.

19. The method as defined in claim 18, wherein the suppressor strain is selected from the group consisting of supE, supP, supD, supU, supF, supZ, supB, supL, supN, supC, supM, glyT, trpT and temperature-sensitive alleles thereof.

20. The method as defined in claim 12, wherein the peptide of interest has therapeutic utility.
